(19) United States
(12) Patent Application Publication
(10) Pub. No.: US 2007/0100192 A1
(43) Pub. Date: May 3, 2007

(54) Olefin isomerization

(76) Inventors: Mark Andrew Harmer, Kennett Square, PA (US); Christopher P. Junk, Wilmington, DE (US); Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/583,185

(22) Filed: Oct. 19, 2006

Related U.S. Application Data
(60) Provisional application No. 60/730,792, filed on Oct. 27, 2005.

(51) Int. Cl. C07C 5/25; C07C 5/23

(52) U.S. Cl. 585/665

(57) ABSTRACT

This invention relates to a process for isomerizing olefins in the presence of at least one ionic liquid.

### FIELD OF THE INVENTION

This invention relates to a process for isomerizing olefins; the reaction is carried out in the presence of at least one ionic liquid.

### BACKGROUND

The isomerization of olefins to internal olefins is an important reaction in the refining industry. Long chain olefins, for example, can be isomerized to the internal olefins, which can be used as precursors to materials used in lubrication.

Various methods for the catalytic isomerization of olefins have been disclosed. See, for example, Dunning, H. N. (Ind. Eng. Chem. (1953) 45:551-564) and U.S. Pat. No. 5,849,974. Homogeneous catalysts have the disadvantage that the product or products from the isomerization reaction require separation from the reaction catalyst. A cost effective, efficient process for the production and purification of olefin isomers is required.

Ionic liquids are liquids composed of ions that are liquid around or below 100° C. (Science (2003) 302:792-793). Ionic liquids exhibit negligible vapor pressure, and with increasing regulatory pressure to limit the use of traditional industrial solvents due to environmental considerations such as volatile emissions and aquifer and drinking water contamination, much research has been devoted to designing ionic liquids that could function as replacements for conventional solvents.

### SUMMARY OF THE INVENTION

The present invention provides a process for carrying out isomerization reactions using ionic liquids as solvent. The use of at least one ionic liquids as the solvent for this reaction allows for ready separation of the product(s) from the catalyst.

The present invention relates to a process for making internal olefins comprising:

(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid comprised of an ammonium cation and an anion having the Formula: 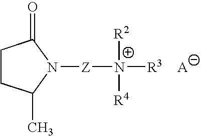
wherein:
(i) Z is —(CH₂)ₙ—, wherein n is an integer from 2 to 12;
(ii) R², R³ and R⁴ taken independently are H, —CH₃, —CH₂CH₃ or C₃ to C₆ straight-chain or branched monovalent alkyl; and
(iii) A⁻ is R⁵—SO₃⁻ or (R⁶—SO₂)₂N⁻; wherein R⁵ and R⁶ are independently selected from the group consisting of:
   (a) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
   (b) —CH₃, —C₂H₅, or C₃ to C₂₅ straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
   (c) C₆ to C₂₅ unsubstituted aryl or unsubstituted heteroaryl having one to three heteroatoms independently selected from the group consisting of O, N and S; and
   (d) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of:
      (1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
      (2) OH,
      (3) NH₂, and
      (4) SH;

thereby forming an isomer phase comprising at least one internal olefin and an ionic liquid phase that comprises the at least one acid catalyst; and

B) separating the isomer phase from the ionic liquid phase, thereby forming a separated ionic liquid phase.

### DETAILED DESCRIPTION OF THE INVENTION

The present invention relates to a process for isomerizing α-olefins in the presence of an ionic liquid solvent. The use of an ionic liquid as the solvent for the isomerization reaction is advantageous because the acid catalyst is recovered in an ionic liquid phase that is separate from the phase comprising the product isomer(s), thus the product isomer(s) are easily separated from the acid catalyst.

Definitions

In this disclosure a number of terms and abbreviations are used. The following definitions are provided.

By “ionic liquid” is meant an organic salt that is liquid around or below 100° C.

By “fluoroalkyl” is meant an alkyl group wherein at least one member selected from the hydrogens has been replaced by fluorine. By “perfluoroalkyl” is meant an alkyl group wherein all of the hydrogens have been replaced by fluorines.

By “alkoxy” is meant a straight-chain or branched alkyl group bound via an oxygen atom. By “fluoroalkoxy” is meant an alkoxy group wherein at least one member selected from the hydrogens has been replaced by fluorine. By “perfluoroalkoxy” is meant an alkoxy group wherein all of the hydrogens have been replaced by fluorines.

By “halogen” is meant bromine, iodine, chlorine or fluorine.

By “heteroaryl” is meant an aryl group having one or more heteroatoms.

By “catalyst” is meant a substance that affects the rate of the reaction but not the reaction equilibrium, and emerges from the process chemically unchanged.

When referring to an alkane, alkene, alkoxy, fluoroalkoxy, perfluoroalkoxy, fluoroalkyl, perfluoroalkyl, aryl or heteroaryl, the term “optionally substituted with at least one member selected from the group consisting of” means that one or more hydrogens on the carbon chain may be independently substituted with one or more of at least one member of the group. For example, substituted C₂H₅ may be, without limitations, CF₂CF₃, CH₂CH₂OH or CF₂CF₂I.

The expression “C₁ to Cn straight-chain or branched”, where n is an integer defining the length of the carbon chain, is meant to indicate that C₁ and C₂ are straight-chain, and C₃ to Cₙ may be straight-chain or branched.

The invention provides a process for making internal olefins comprising:

(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid comprised of an ammonium cation and an anion having the Formula: 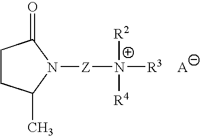
wherein:
(i) Z is —(CH₂)ₙ—, wherein n is an integer from 2 to 12;
(ii) R², R³ and R⁴ taken independently are H, —CH₃, —CH₂CH₃ or C₃ to C₆ straight-chain or branched monovalent alkyl; and
(iii) A⁻ is R⁵—SO₃⁻ or (R⁶—SO₂)₂N⁻; wherein R⁵ and R⁶ are independently selected from the group consisting of:
   (a) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
   (b) —CH₃, —C₂H₅, or C₃ to C₂₅ straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
   (c) C₆ to C₂₅ unsubstituted aryl or unsubstituted heteroaryl having one to three heteroatoms independently selected from the group consisting of O, N and S; and
   (d) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of:
      (1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
      (2) OH,
      (3) NH₂, and
      (4) SH;

thereby forming an isomer phase comprising at least one internal olefin and an ionic liquid phase that comprises the at least one acid catalyst; and

B) separating the isomer phase from the ionic liquid phase, thereby forming a separated ionic liquid phase.

In another more specific embodiment, A⁻ is selected from the group consisting of: [CH₃OSO₃]⁻, [C₂H₅OSO₃]⁻, [CF₃SO₃]⁻, [HCF₂CF₂SO₃]⁻, [CF₃HFCCF₂SO₃]⁻, [HCCIFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃], [CF₃CFHOCF₂CF₂SO₃]⁻, [CF₂HCF₂OCF₂CF₂SO₃]⁻, [CF₂ICF₂OCF₂CF₂SO₃], [CF₃CF₂OCF₂CF₂SO₃]⁻, and [(CF₂HCF₂SO₂)₂N]⁻, [(CF₃CFHCF₂SO₂)₂N]⁻.

In one more specific embodiment, n of the ionic liquid is from 2 to 6.

The α-olefin starting material comprises from about four carbons to about twenty five carbons. In a more specific embodiment, the α-olefin starting material may comprise from about 12 carbons to about 18 carbons. The starting material may comprise either linear or branched olefins, however preferably the starting material will comprise greater than 60 mol % linear α-olefin. The starting material may also comprise from about 10 mol % to about 35 mol % branched α-olefin, from about 0 mol % to about 10 mol % linear internal olefin, and/or from about 0 mol % to about 10 mol % branched internal olefin. The olefin starting material may also be admixed with one or more inert hydrocarbons, such as paraffins, cycloparaffins, or aromatics, however preferably, the olefin starting material comprises at least 90% by weight of olefins.

The at least one acid catalyst usable in the current process is selected from the group consisting of rare earth fluorinated alkyl sulfonates, organic sulfonic acids, fluoroalkyl sulfonic acids, metal sulfonates, metal trifluoroacetates, and combinations thereof.

In a preferred embodiment, the at least one acid catalyst is selected from the group consisting of:

(i) bismuth triflate;

(ii) yttrium triflate;

(iii) ytterbium triflate;

(iv) neodymium triflate;

(v) lanthanum triflate;

(vi) scandium triflate;

(vii) zirconium triflate;

(viii) Formula (I); 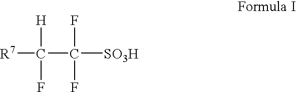
wherein:
R⁷ is selected from the group consisting of:
(1) halogen;
(2) —CH₃, —C₂H₅ or C₃ to C₁₅, preferably C₃ to C₆, straight-chain or branched alkane or alkene, optionally substituted with at least one member selected from the group consisting of Cl, Br, I, OH, NH₂ and SH;
(3) —OCH₃, —OC₂H₅ or C₃ to C₁₅, preferably C₃ to C₆, straight-chain or branched alkoxy, optionally substituted with at least one member selected from the group consisting of Cl, Br, I, OH, NH₂ and SH;
(4) C₁ to C₁₅, preferably C₃ to C₆, straight-chain or branched fluoroalkyl, optionally substituted with at least one member selected from the group consisting of Cl, Br, I, OH, NH₂ and SH;
(5) C₁ to C₁₅, preferably C₃ to C₆, straight-chain or branched fluoroalkoxy, optionally substituted with at least one member selected from the group consisting of Cl, Br, I, OH, NH₂ and SH;
(6) C₁ to C₁₅, preferably C₃ to C₆, straight-chain or branched perfluoroalkyl; and
(7) C₁ to C₁₅, preferably C₃ to C₆, straight-chain or branched perfluoroalkoxy;

(ix) Formula (II) 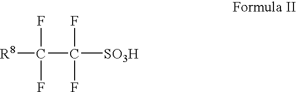
wherein:
R⁸ is selected from the group consisting of:
(1) —CH₃, —C₂H₅ or C₃ to C₁₅, preferably C₃ to C₆, straight-chain or branched alkoxy, optionally substituted with at least one member selected from the group consisting of Cl, Br, I, OH, NH₂ and SH;
(2) C₁ to C₁₅, preferably C₃ to C₆, straight-chain or branched fluoroalkoxy, optionally substituted with at least one member selected from the group consisting of Cl, Br, I, OH, NH₂ and SH; and
(3) C₁ to C₁₅, preferably C₃ to C₆, straight-chain or branched perfluoroalkoxy; and

(x) Formula (III); 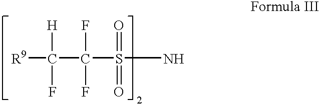
wherein:
R⁹ is selected from the group consisting of:
(1) halogen;
(2) —CH₃, —C₂H₅ or C₃ to C₁₅, preferably C₃ to C₆, straight-chain or branched alkane or alkene, optionally substituted with at least one member selected from the group consisting of Cl, Br, I, OH, NH₂ and SH;

(3) —OCH₃, —OC₂H₅ or C₃ to C₁₅, preferably C₃ to C₆, straight-chain or branched alkoxy, optionally substituted with at least one member selected from the group consisting of Cl, Br, I, OH, NH₂ and SH;

(4) C₁ to C₁₅, preferably C₃ to C₆, straight-chain or branched fluoroalkyl, optionally substituted with at least one member selected from the group consisting of Cl, Br, I, OH, NH₂ and SH;

(5) C₁ to C₁₅, preferably C₃ to C₆, straight-chain or branched fluoroalkoxy, optionally substituted with at least one member selected from the group consisting of Cl, Br, I, OH, NH₂ and SH;

(6) C₁ to C₁₅, preferably C₃ to C₆, straight-chain or branched perfluoroalkyl; and

(7) C₁ to C₁₅, preferably C₃ to C₆, straight-chain or branched perfluoroalkoxy.

In a more specific embodiment, the at least one acid catalyst is 1,1,2,2-tetrafluoroethanesulfonic acid, 1,1,2,3,3,3-hexafluoropropanesulfonic acid, 2-chloro-1,1,2-trifluoroethanesulfonic acid, 1,1,2-trifluoro-2-(perfluoroethoxy)ethanesulfonic acid, 1,1,2-trifluoro-2-(trifluoromethoxy)ethanesulfonic acid, or 1,1,2-trifluoro-2-(perfluoropropoxy)ethanesulfonic acid.

Most of the catalysts may be obtained commercially. The catalysts not available commercially may be synthesized as described in the following references: U.S. Pat. No. 2,403,207, Rice, et al. (Inorg. Chem., 1991, 30:4635-4638), Coffman, et al. (J. Org. Chem., 1949, 14:747-753 and Koshar, et al. (supra).

The at least one acid catalyst is used at a concentration of from about 0.1% to about 20% by weight of the total weight of the α-olefin(s) at the start of the reaction. In a more specific embodiment, the at least one acid catalyst is used at a concentration of from about 0.1% to about 5% by weight of the total weight of the α-olefin(s) at the start of the reaction.

The reaction is preferably carried out at a temperature of from about 50° C. to about 175° C. In a more specific embodiment, the reaction is carried out at a temperature of from about 50° C. to about 120° C.

The reaction is preferably carried out under an inert atmosphere, such as nitrogen, argon or helium. The reaction may be performed at atmospheric pressure, or at pressures above atmospheric pressure.

The time for the reaction will depend on many factors, such as the reactants, reaction conditions and reactor. One skilled in the art will know to adjust the time for the reaction to achieve optimal isomerization of the α-olefins.

The ionic liquid useful for the invention may be synthesized from a pyrrolidine-2-one of the Formula: 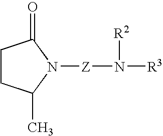
wherein:
(i) Z is —(CH₂)ₙ—, wherein n is an integer from 2 to 12; and
(ii) R² and R³ taken independently are H, —CH₃, —CH₂CH₃ or C₃ to C₆ straight-chain or branched monovalent alkyl.

### Synthesis of N-hydrocarbyl pyrrolidine-2-one

The pyrrolidine-2-one may be synthesized by contacting levulinic acid or an ester thereof with a diamine of the Formula R²R³N-Z-NH₂ in the presence of hydrogen gas and a catalyst according to Reaction (I): 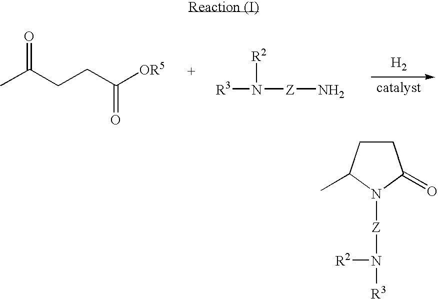
wherein:
(i) Z is —(CH₂)ₙ—, wherein n is an integer from 2 to 12;
(ii) R² and R³ taken independently are H, —CH₃, —CH₂CH₃ or C₃ to C₆ straight-chain or branched monovalent alkyl; and
(iii) R⁵ is H, —CH₃, —CH₂CH₃ or C₃ to C₈ straight-chain or branched monovalent alkyl.

In another embodiment, the pyrrolidine-2-one may be synthesized by contacting a salt of levulinic acid, such as ammonium levulinate, with a diamine of the Formula R²R³N-Z-NH₂ in the presence of hydrogen gas and a catalyst.

The pyrrolidine-2-one formed in Reaction (I) can be synthesized according to the methods and conditions taught in U.S. Pat. No. 6,818,593 (hereinafter to referred to as '593). Although '593 describes the synthesis of 5-methyl-N-alkyl-2-pyrrolidinone from the reductive amination of levulinic acid with nitro compounds, the methods and conditions taught in '593 (column 2, line 66 through column 7, line 21) can be utilized for the process described by Reaction (I) wherein levulinic acid, a salt thereof, or an ester thereof and a diamine are converted to a pyrrolidine-2-one in the presence of hydrogen gas and a catalyst.

Levulinic acid may be obtained from biomass. For the conversion of biomass to levulinic acid, biomass may be contacted with water and an acid catalyst in a train of one or more reactors, preferably under pressure at elevated temperature. This basic process is described, for example, in U.S. Pat. No. 5,608,105, U.S. Pat. No. 5,859,263, U.S. Pat. No. 6,054,611 and U.S. Patent Application 2003/0233011. Generally, cellulose in the biomass is converted to levulinic acid and formate in one or more reactors. Levulinic acid produced from biomass may also be converted to levulinic acid esters for example as described in U.S. 2003/0233011A1through the reaction of levulinic acid with olefins.

For the synthesis of pyrrolidine-2-ones according to Reaction (I), a molar ratio of diamine to levulinic acid, a salt thereof, or an ester thereof of from about 0.01/1 to about 100/1 is preferred at the start of the reaction; a molar ratio of about 0.3/1 to about 5/1 is further preferred at the start of the reaction. A temperature range of from about 25° C. to about 300° C. is used for the reductive amination reaction; a temperature range of from about 75° C. to about 200° C. is preferred. A pressure range of from about 0.3 MPa to about 20.0 MPa is employed for the reaction; a pressure range of from about 1.3 MPa to about 7.6 MPa is preferred. The reaction may be performed in a non-reacting solvent medium such as water, alcohols, ethers, and pyrrolidones. Alternatively, the excess of diamine can also act as the medium of the reaction.

The principal component of the catalyst useful for Reaction (I) is selected from metals from the group consisting of palladium, ruthenium, rhenium, rhodium, iridium, platinum, nickel, cobalt, copper, iron, osmium; and combinations thereof.

A chemical promoter may augment the activity of a catalyst. The promoter may be incorporated into the catalyst during any step in the chemical processing of the catalyst constituent. The chemical promoter generally enhances the physical or chemical function of the catalyst agent, but can also be added to retard undesirable side reactions. Suitable promoters useful to prepare the ionic liquid useful for the processes of the invention include metals selected from tin, zinc, copper, gold, silver, and combinations thereof. The preferred metal promoter is tin. Other promoters that can be used are elements selected from Group 1 and Group 2 of the Periodic Table.

The catalyst may be supported or unsupported. A supported catalyst is one in which the active catalyst agent is deposited on a support material by a number of methods, such as spraying, soaking or physical mixing, followed by drying, calcination, and if necessary, activation through methods such as reduction or oxidation. Materials frequently used as a support are porous solids with high total surface areas (external and internal) which can provide high concentrations of active sites per unit weight of catalyst. The catalyst support may enhance the function of the catalyst agent. A supported metal catalyst is a supported catalyst in which the catalyst agent is a metal.

A catalyst that is not supported on a catalyst support material is an unsupported catalyst. An unsupported catalyst may be platinum black or a Raney® (W.R. Grace & Co., Columbia, Md.) catalyst. Raney® catalysts have a high surface area due to selectively leaching an alloy containing the active metal(s) and a leachable metal (usually aluminum). Raney® catalysts have high activity due to the higher specific area and allow the use of lower temperatures in hydrogenation reactions. The active metals of Raney® catalysts include nickel, copper, cobalt, iron, rhodium, ruthenium, rhenium, osmium, iridium, platinum, palladium; and combinations thereof.

Promoter metals may also be added to the base Raney® metals to affect selectivity and/or activity of the Raney® catalyst. Promoter metals for Raney® catalysts may be selected from transition metals from Groups IIIA through VIIIA, IB and IIB of the Periodic Table of the Elements. Examples of promoter metals include chromium, molybdenum, platinum, rhodium, ruthenium, osmium, and palladium, typically at about 2% by weight of the total metal.

The catalyst support useful herein can be any solid, inert substance including, but not limited to, oxides such as silica, alumina and titania; barium sulfate; calcium carbonate; and carbons. The catalyst support can be in the form of powder, granules, pellets, or the like.

A preferred support material of the invention is selected from the group consisting of carbon, alumina, silica, silica-alumina, silica-titania, titania, titania-alumina, barium sulfate, calcium carbonate, strontium carbonate, and combinations thereof. Supported metal catalysts can also have supporting materials made from one or more compounds. More preferred supports are carbon, titania and alumina. Further preferred supports are carbons with a surface area greater than 100 m²/g. A further preferred support is carbon with a surface area greater than 200 m²/g. Preferably, the carbon has an ash content that is less than 5% by weight of the catalyst support; the ash content is the inorganic residue (expressed as a percentage of the original weight of the carbon) which remains after incineration of the carbon.

The preferred content of the metal catalyst in the supported catalyst is from about 0.1% to about 20% of the supported catalyst based on metal catalyst weight plus the support weight. A more preferred metal catalyst content range is from about 1% to about 10% of the supported catalyst.

Combinations of metal catalyst and support system may include any one of the metals referred to herein with any of the supports referred to herein. Preferred combinations of metal catalyst and support include palladium on carbon, palladium on calcium carbonate, palladium on barium sulfate, palladium on alumina, palladium on titania, platinum on carbon, platinum on alumina, platinum on silica, iridium on silica, iridium on carbon, iridium on alumina, rhodium on carbon, rhodium on silica, rhodium on alumina, nickel on carbon, nickel on alumina, nickel on silica, rhenium on carbon, rhenium on silica, rhenium on alumina, ruthenium on carbon, ruthenium on alumina and ruthenium on silica.

Further preferred combinations of metal catalyst and support include palladium on carbon, palladium on alumina, palladium on titania, platinum on carbon, platinum on alumina, rhodium on carbon, rhodium on alumina, ruthenium on carbon and ruthenium on alumina.

Suitable diamines for Reaction (I) may be obtained commercially from, for example, Huntsman (Houston, Tex.) or BASF (Mount Olive, N.J.), or may be synthesized by methods well known to those skilled in the art. For a discussion of the synthesis of diamines, see, for example, Eller, K. and Henkes, E., Diamines and Polyamines (Ullmann's Encyclopedia of Industrial Chemistry (2002) Wiley-VCH Verlag GmbH & Co, Chapter 8) and Experimental Methods in Organic Chemistry, 3^{rd} Edition (Moore, J., Dalrymple, D. and Rodig, O. (eds.) (1982) Saunders College Publishing, NY, Chapter 22. Suitable diamines are those having the Formula R²R³N-Z-NH₂ wherein Z is —(CH₂)ₙ—, wherein n is an integer from 2 to 12 and R² and R³ taken independently are H, —CH₃, —CH₂CH₃ or C₃ to C₆ straight-chain or branched monovalent alkyl.

The formation of pyrrolidine-2-ones may be carried out in batch, sequential batch (i.e., a series of batch reactors) or in continuous mode in any of the equipment customarily employed for continuous process (see for example, H. S. Fogler, Elementary Chemical Reaction Engineering, Prentice-Hall, Inc., N.J., USA).

The pyrrolidinones synthesized according to Reaction (I) may be recovered, for example, by distillation, or by filtration to remove solid acid catalyst particles if present.

### Conversion of the pyrrolidine-2-one to an Ionic Liquid

The non-ring nitrogen of the pyrrolidine-2-one is quaternized to obtain an ammonium compound of the Formula: 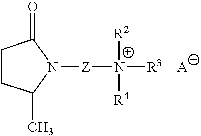
wherein Z is —(CH₂)ₙ— wherein n is an integer from 2 to 12, R², R³, and R⁴ taken independently are —CH₃, —CH₂CH₃ or C₃ to C₆ straight-chain or branched monovalent alkyl, and A- is selected from the group consisting of Cl⁻, Br⁻, and I⁻.

In order to form the ionic liquid, the pyrrolidine-2-one is contacted with an alkylating halide having the Formula R¹-A wherein R¹ is selected from the group consisting of —CH₃, —CH₂CH₃ or C₃ to C₆ straight-chain or branched monovalent alkyl, and A⁻ is selected from the group consisting of Cl⁻, Br⁻, and I⁻. Methods for performing quaternization reactions are well-known and are described in Organic Chemistry (Morrison and Boyd (ed.) 3^{rd} Edition (1973) Allyn and Bacon, Inc., Boston, Chapter 23.5, pages 752-753).

The quaternization reaction may optionally be carried out in an inert solvent, such as acetonitrile, acetone or dichloromethane. The quaternization may be accomplished by refluxing of the reactants, optionally under an inert atmosphere. When the compositions of the present invention and/or the reactants used for synthesis of the compositions are hygroscopic, it is preferable to carry out the quaternization and/or anion exchange reaction (see below) under conditions that exclude water and air. The alkylating halide is present in slight excess (ca. 5%) at the start of the reaction. The reaction is carried out at a temperature of from about 10° C. to about 80° C.; the reaction is preferably carried out at a temperature of from about 30° C. to about 70° C., more preferably from about 60° C. to about 70° C. The time for the reaction is generally from about 1 minute to about 48 hours; the time for the reaction is preferably from about 30 minutes to about 24 hours. This product, though an ionic liquid, is not suitable for the process of this invention. The anion is halide, a stronger base than the anion of the acid catalyst of the invention. The presence of halide would act to reduce the acid strength, weakening the catalytic effect.

Anion Exchange

The ionic liquid may be converted to a different ionic liquid suitable for the process of the invention by an anion exchange reaction. Thus, the quaternary ammonium compound (ionic liquid) is contacted with M⁺A⁻, wherein M is selected from the group consisting of H, Li, K, Na, Ag, Mg, Ca, Ce, Ba, Rb and Sr, and A⁻ is an anion as described earlier, to form a composition having the desired anion.

Prior to the exchange reaction, excess alkylating agent may be removed, for example, by evaporation. In addition, the quaternary ammonium compound may be washed with a solvent and dried prior to the anion exchange reaction. The anion exchange reaction may be carried out by mixing the quaternary ammonium compound with M⁺A⁻, optionally under an inert atmosphere. The anion exchange reaction may be carried out at a temperature of from about −20° C. to about 100° C. for a time of about 1 second to about 72 hours. Solvents useful in the reaction should be inert to the reactants and products, and include methanol, ethanol, acetone and acetonitrile. Choice of the appropriate solvent or mixture of solvents will allow for separation of the composition comprising the desired anion from the composition comprising the less desired anion as is well known in the art. Additional techniques may be utilized to enhance the anion exchange reaction, such as ultrasonication as taught in WO 03/048078.

The anions of the ionic liquid may be obtained commercially, or may be synthesized according to methods known in the art. The fluoroalkyl sulfonate anions of the anions may be synthesized from perfluorinated terminal olefins or perfluorinated vinyl ethers generally according to the method of Koshar, et al. (J. Am. Chem. Soc. (1953) 75:4595-4596); in one embodiment, sulfite and bisulfite are used as the buffer in place of bisulfite and borax, and in another embodiment, the reaction is carried in the absence of a radical initiator. 1,1,2,2-Tetrafluoroethanesulfonate, 1,1,2,3,3,3-hexafluoropropanesulfonate, 1,1,2-trifluoro-2-(trifluoromethoxy)ethanesulfonate, 1,1,2-trifluoro-2-(pentafluoroethoxy)ethanesulfonate, and 1,1,2-trifluoro-2-(perfluoropropoxy)ethanesulfonate, may be synthesized according to Koshar, et al. (supra), with modifications. Preferred modifications include using a mixture of sulfite and bisulfite as the buffer, freeze drying or spray drying to isolate the crude 1,1,2,2-tetrafluoroethanesulfonate and 1,1,2,3,3,3-hexafluoropropanesulfonate products from the aqueous reaction mixture, using acetone to extract the crude 1,1,2,2-tetrafluoroethanesulfonate and 1,1,2,3,3,3-hexafluoropropanesulfonate salts, and crystallizing 1,1,2-trifluoro-2-(trifluoromethoxy)ethanesulfonate, 1,1,2-trifluoro-2-(pentafluoroethoxy)ethanesulfonate, and 1,1,2-perfluoro-2-(trifluoropropoxy)ethanesulfonate, from the reaction mixture by cooling.

The physical and chemical properties of ionic liquids can be specifically selected by choice of the appropriate cation and anion. For example, increasing the chain length of one or more alkyl chains of the cation will affect properties such as the melting point, hydrophilicity/lipophilicity, density and solvation strength of the ionic liquid. Choice of the anion can affect, for example, the melting point, the water solubility and the acidity and coordination properties of the composition. Effects of cation and anion on the physical and chemical properties of ionic liquids are known to those skilled in the art and are reviewed in detail by Wasserscheid and Keim (Angew. Chem. Int. Ed. (2000) 39:3772-3789) and Sheldon (Chem. Commun. (2001) 2399-2407). In the present invention, the choice of the ionic liquid may affect the degree of formation of internal olefins. In addition, the ionic liquid can increase the activity of the catalyst.

The process of the present invention may be carried out in batch, sequential batch (i.e., a series of batch reactors) or in continuous mode in any of the equipment customarily employed for continuous process (see for example, Fogler, supra).

An advantage to the use of at least one ionic liquid in this reaction is that the reaction product comprises an olefin phase comprising the internal olefin(s) and an ionic liquid phase that comprises the acid catalyst. Thus the internal olefin(s) is/are easily recoverable from the acid catalyst by, for example, decantation. In a preferred embodiment, the separated ionic liquid phase is reused to form the reaction mixture.

### EXAMPLES

### General Materials and Methods

The following abbreviations are used:

gas chromatography is abbreviated GC, Centigrade is abbreviated C, gram is abbreviated g, kilogram is abbreviated kg, milliliter(s) is abbreviated ml, weight percent is abbreviated wt %; milliequivalents is abbreviated meq; and melting point is abbreviated Mp.

1-Dodecene and iodopropane were obtained from Aldrich (St. Louis, Mo.). Octadecyl-3-methylimidazolium chloride, 1-dodecyl-3-methyl imidazolium chloride, tetrahydrofuran, iodopropane, acetonitrile, iodoperfluorohexane, toluene, 1,3-propanediol, oleum (20% SO₃), sodium sulfite (Na₂SO₃, 98%), and acetone were obtained from Acros (Hampton, N.H.). Potassium metabisulfite (K₂S₂O₅, 99%), was obtained from Mallinckrodt Laboratory Chemicals (Phillipsburg, N.J.). Potassium sulfite hydrate (KHSO₃.xH₂O, 95%), sodium bisulfite (NaHSO₃), sodium carbonate, magnesium sulfate, ethyl ether, trioctyl phosphine, and 1-ethyl-3-methylimidazolium chloride (98%) were obtained from Aldrich (St. Louis, Mo.). Sulfuric acid and methylene chloride were obtained from EMD Chemicals, Inc. (Gibbstown, N.J.). Perfluoro(ethyl vinyl ether), perfluoro(methyl vinyl ether), hexafluoropropene and tetrafluoroethylene were obtained from DuPont Fluoroproducts (Wilmington, Del.). 1-Butyl-methylimidazolium chloride was obtained from Fluka (Sigma-Aldrich, St. Louis, Mo.). Tetra-n-butylphosphonium bromide and tetradecyl(tri-n-hexyl)phosphonium chloride were obtained from Cytec Canada Inc. (Niagara Falls, Ontario, Canada). 1,1,2,2-Tetrafluoro-2-(pentafluoroethoxy)sulfonate was obtained from SynQuest Laboratories, Inc. (Alachua, Fla.). ESCAT-142 (Pd/C catalyst) was obtained from Engelhard Corp. (Iselin, N.J.). Ethyl levulinate and N,N-dimethylethylenediamine were obtained from Alfa Aesar (Ward Hill, Mass.).

### Preparation of Anions not Generally Available Commercially

### (A) Synthesis of potassium 1,1,2,2-tetrafluoroethanesulfonate (TFES-K)

A 1-gallon Hastelloy® C276 reaction vessel was charged with a solution of potassium sulfite hydrate (176 g, 1.0 mol), potassium metabisulfite (610 g, 2.8 mol) and deionized water (2000 ml). The pH of this solution was 5.8. The vessel was cooled to 18° C., evacuated to 0.10 MPa, and purged with nitrogen. The evacuate/purge cycle was repeated two more times. To the vessel was then added tetrafluoroethylene (TFE, 66 g), and it was heated to 100° C. at which time the inside pressure was 1.14 MPa. The reaction temperature was increased to 125° C. and kept there for 3 hr. As the TFE pressure decreased due to the reaction, more TFE was added in small aliquots (20-30 g each) to maintain operating pressure roughly between 1.14 and 1.48 MPa. Once 500 g (5.0 mol) of TFE had been fed after the initial 66 g precharge, the vessel was vented and cooled to 25° C. The pH of the clear light yellow reaction solution was 10-11. This solution was buffered to pH 7 through the addition of potassium metabisulfite (16 g).

The water was removed in vacuo on a rotary evaporator to produce a wet solid. The solid was then placed in a freeze dryer (Virtis Freezemobile 35xl; Gardiner, N.Y.) for 72 hr to reduce the water content to approximately 1.5 wt % (1387 g crude material). The theoretical mass of total solids was 1351 g. The mass balance was very close to ideal and the isolated solid had slightly higher mass due to moisture. This added freeze drying step had the advantage of producing a free-flowing white powder whereas treatment in a vacuum oven resulted in a soapy solid cake that was very difficult to remove and had to be chipped and broken out of the flask.

The crude TFES-K can be further purified and isolated by extraction with reagent grade acetone, filtration, and drying.

¹⁹F NMR (D₂O) □-122.0 (dt, J_{FH}=6 Hz, J_{FF}=6 Hz, 2F); −136.1 (dt, J_{FH}=53 Hz, 2F). ¹H NMR (D₂O) □6.4 (tt, J_{FH}=53 Hz, J_{FH}=6 Hz, 1H). % Water by Karl-Fisher titration: 580 ppm. Analytical calculation for C₂HO₃F₄SK: C, 10.9; H, 0.5; N, 0.0. Experimental results: C, 11.1; H, 0.7; N, 0.2. Mp (DSC): 242° C. TGA (air): 10% wt. loss @ 367° C., 50% wt. loss @ 375° C. TGA (N₂): 10% wt. loss @ 363° C., 50% wt. loss @ 375° C.

### (B) Synthesis of potassium-1,1,2-trifluoro-2-(perfluoroethoxy)ethanesulfonate (TPES-K)

A 1-gallon Hastelloy® C276 reaction vessel was charged with a solution of potassium sulfite hydrate (88 g, 0.56 mol), potassium metabisulfite (340 g, 1.53 mol) and deionized water (2000 ml). The vessel was cooled to 7° C., evacuated to 0.05 MPa, and purged with nitrogen. The evacuate/purge cycle was repeated two more times. To the vessel was then added perfluoro(ethyl vinyl ether) (PEVE, 600 g, 2.78 mol), and it was heated to 125° C. at which time the inside pressure was 2.31 MPa. The reaction temperature was maintained at 125° C. for 10 hr. The pressure dropped to 0.26 MPa at which point the vessel was vented and cooled to 25° C. The crude reaction product was a white crystalline precipitate with a colorless aqueous layer (pH=7) above it.

The ¹⁹F NMR spectrum of the white solid showed pure desired product, while the spectrum of the aqueous layer showed a small but detectable amount of a fluorinated impurity. The desired isomer is less soluble in water so it precipitated in isomerically pure form.

The product slurry was suction filtered through a fritted glass funnel, and the wet cake was dried in a vacuum oven (60° C., 0.01 MPa) for 48 hr. The product was obtained as off-white crystals (904 g, 97% yield).

¹⁹F NMR (D₂O) δ −86.5 (s, 3F); −89.2, −91.3 (subsplit ABq, J_{FF}=147 Hz, 2F); −119.3, −121.2 (subsplit ABq, J_{FF}=258 Hz, 2F); −144.3 (dm, J_{FH}=53 Hz, 1F). ¹H NMR (D₂O) δ 6.7 (dm, J_{FH}=53 Hz, 1H). Mp (DSC) 263° C. Analytical calculation for C₄HO₄F₈SK: C, 14.3; H, 0.3 Experimental results: C, 14.1; H, 0.3. TGA (air): 10% wt. loss @ 359° C., 50% wt. loss @ 367° C. TGA (N₂): 10% wt. loss @ 362° C., 50% wt. loss @ 374° C.

### (C) Synthesis of potassium-1,1,2-trifluoro-2-(trifluoromethoxy)ethanesulfonate (TTES-K)

A 1-gallon Hastelloy® C276 reaction vessel was charged with a solution of potassium sulfite hydrate (114 g, 0.72 mol), potassium metabisulfite (440 g, 1.98 mol) and deionized water (2000 ml). The pH of this solution was 5.8. The vessel was cooled to −35° C., evacuated to 0.08 MPa, and purged with nitrogen. The evacuate/purge cycle was repeated two more times. To the vessel was then added perfluoro(methyl vinyl ether) (PMVE, 600 g, 3.61 mol) and it was heated to 125° C. at which time the inside pressure was 3.29 MPa. The reaction temperature was maintained at 125° C. for 6 hr. The pressure dropped to 0.27 MPa at which point the vessel was vented and cooled to 25° C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity.

The solution was suction filtered through a fritted glass funnel for 6 hr to remove most of the water. The wet cake was then dried in a vacuum oven at 0.01 MPa and 50° C. for 48 hr. This gave 854 g (83% yield) of a white powder. The final product was isomerically pure (by ¹⁹F and ¹H NMR) since the undesired isomer remained in the water during filtration.

¹⁹F NMR (D₂O) δ −59.9 (d, J_{FH}=4 Hz, 3F); −119.6, −120.2 (subsplit ABq, J=260 Hz, 2F); −144.9 (dm, J_{FH}=53 Hz, 1F). ¹H NMR (D₂O) δ 6.6 (dm, J_{FH}=53 Hz, 1H). % Water by Karl-Fisher titration: 71 ppm. Analytical calculation for C₃HF₆SO₄K: C, 12.6; H, 0.4; N, 0.0. Experimental results: C, 12.6; H, 0.0; N, 0.1. Mp (DSC) 257° C. TGA (air): 10% wt. loss @ 343° C., 50% wt. loss @ 358° C. TGA (N₂): 10% wt. loss @ 341° C., 50% wt. loss @ 357° C.

### (D) Synthesis of sodium 1,1,2,3,3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 ml). The pH of this solution was 5.7. The vessel was cooled to 4° C., evacuated to 0.08 MPa, and then charged with hexafluoropropene (HFP, 120 g, 0.8 mol, 0.43 MPa). The vessel was heated with agitation to 120° C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140° C., 48 hr) to produce 219 g of white solid which contained approximately 1 wt % water. The theoretical mass of total solids was 217 g.

The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F). ¹H NMR (D₂O) δ 5.8 (dm, J_{FH}=43 Hz, 1H). Mp (DSC) 126° C. TGA (air): 10% wt. loss @ 326° C., 50% wt. loss @ 446° C. TGA (N₂): 10% wt. loss @ 322° C., 50% wt. loss @ 449° C.

### Preparation of Catalysts not Generally Available Commercially

### (E) Synthesis of 1,1,2,2-tetrafluoroethanesulfonic acid (TFESA)

A 100 ml round bottomed flask with a sidearm and equipped with a digital thermometer and magnetic stirr bar was placed in an ice bath under positive nitrogen pressure. To the flask was added 50 g crude TFES-K (from synthesis (A) above), 30 g of concentrated sulfuric acid (95-98%) and 78 g oleum (20 wt % SO₃) while stirring. The amount of oleum was chosen such that there would be a slight excess of SO₃ after the SO₃ reacted with and removed the water in the sulfuric acid and the crude TFES-K. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump (Buchi V-500, Buchi Analytical, Inc., Wilmington, Del.) in steps of 100 Torr (13 kPa) in order to avoid foaming. A dry-ice trap was placed between the distillation apparatus and the pump to collect any excess SO₃. When the pot temperature reached 120° C. and the pressure was held at 20-30 Torr (2.7-4.0 kPa) a colorless liquid started to reflux which distilled at 110° C. and 31 Torr (4.1 kPa). A forerun of lower-boiling impurity (2.0 g) was obtained before collecting 28 g of the desired colorless acid, TFESA.

It was calculated that approximately 39.8 g TFES-K was present in the 50 g of impure TFES-K. Thus, the 28 g of product is an 85% yield of TFESA from TFES-K, as well as an 85% overall yield from TFE. Analysis gave the following results: 19F NMR (CD₃OD) −125.2 dt, 3JFH=6 Hz, 3JFF=8 Hz, 2F); −137.6 (dt, 2JFH=53 Hz, 2F). 1H NMR (CD3OD) 6.3 (tt, 3JFH=6 Hz, 2JFH=53 Hz, 1H).

### (F) Synthesis of 1,1,2,3,3,3-hexfluoropropanesulfonic acid (HFPSA)

A 100 ml round bottomed flask with a sidearm and equipped with a digital thermometer and magnetic stirr bar was placed in an ice bath under positive nitrogen pressure. To the flask was added 50 g crude sodium hexafluoropropanesulfonate (HFPS-Na) (from synthesis (D) above), 30 g of concentrated sulfuric acid (95-98%) and 58.5 g oleum (20 wt % SO₃) while stirring.

The amount of oleum was chosen such that there would be a slight excess of SO₃ after the SO₃ reacted with and removed the water in the sulfuric acid and the crude HFPSA. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump in steps of 100 Torr (13 kPa) in order to avoid foaming. A dry-ice trap was placed between the distillation apparatus and the pump to collect any excess SO₃. When the pot temperature reached 100° C. and the pressure was held at 20-30 Torr (2.7-4 kPa) a colorless liquid started to reflux and later distilled at 118° C. and 23 Torr (3.1 kPa). A forerun of lower-boiling impurity (1.5 g) was obtained before collecting 36.0 g of the desired acid, hexafluoropropanesulfonic acid (HFPS).

It was calculated that approximately 44 g HFPS-Na was present in 50 g of impure HFPS-Na. Thus, the 36.0 g of HFPSA product was an 89% yield from HFPS-Na, as well as an 84% overall yield from HFP.

¹⁹F NMR (D₂O) −74.5 m, 3F); −113.1, −120.4 (ABq, J=264 Hz, 2F); −211.6 (dm, 1F). ¹H NMR (D₂O) 5.8 (dm, 2JFH=43 Hz, 1H).

### Preparation of Ionic Liquids

### Synthesis of the Ionic Liquid 1-(2-N,N,N-dimethylpropylaminoethyl)-5-methyl pyrrolidine-2-one 1,1,2-trifluoro-2-(perfluoroethoxy)ethanesulfonate

Ethyl levulinate (18.5 g), N,N-dimethylethylenediamine (11.3 g), and 5% Pd/C (ESCAT-142, 1.0 g) were mixed in a 400 ml shaker tube reactor. The reaction was carried out at 150° C. for 8 hr under 6.9 MPa of H₂. The reactants and products were analyzed by gas chromatography on a HP-6890 GC (Agilent Technologies; Palo Alto, Calif.) and HP-5972A GC-MS detector equipped with a 25M×0.25 MM ID CP-Wax 58 (FFAP) column. The GC yields were obtained by adding methoxyethyl ether as the internal standard. The ethyl levulinate conversion was 99.7%, and the product selectivity for 1-(2-N,N-dimethylaminoethyl)-5-methyl-pyrrolidine-2-one was 96%.

For the quaternization reaction, purified 1-(2-N,N-dimethylaminoethyl)-5-methyl-pyrrolidine-2-one (1.7 g) was placed in 5 g of dry acetonitrile, and 1.69 g of 1-iodopropane was added. This mixture was refluxed overnight under a nitrogen atmosphere; the reaction was shown to be complete via TLC, yielding the iodide salt of the quaternary ammonium compound. The acetonitrile was then removed under vacuum.

The ionic liquid 1-(2-N,N,N-dimethylpropylaminoethyl)-5-methyl pyrrolidine-2-one 1,1,2-trifluoro-2-(perfluoroethoxy)ethanesulfonate is prepared by reacting 1-(2-N,N,N-dimethylpropylaminoethyl)-5-methyl pyrrolidine-2-one with 1,1,2-trifluoro-2-(perfluoroethoxy)ethanesulfonate. 1-(2-N,N,N-Dimethylpropylaminoethyl)-5-methyl-pyrrolidine-2-one iodide (3 g) is added to 20 mls of acetone, and an equimolar amount of potassium 1,1,2-trifluoro-2-(perfluoroethoxy)ethanesulfonate is added. The mixture is heated for 17 hours at 60° C.; the solvent is removed by heating, and the solid potassium iodide is removed by filtration, yielding the ionic liquid.

### Example 1

### Isomerization of 1-dodecene in the Presence of the Ionic Liquid 1-(2-N,N,N-dimethylpropylaminoethyl)-5-methyl pyrrolidine-2-one 1,1,2-trifluoro-2-(perfluoroethoxy)ethanesulfonate

The ionic liquid 1-(2-N,N,N-dimethylpropylaminoethyl)-5-methyl pyrrolidine-2-one 1,1,2-trifluoro-2-(perfluoroethoxy)ethanesulfonate (2.0 g) is weighed into a small round-bottomed flask, and the flask is dried overnight at 150° C. under vacuum. The flask is removed from the oven, quickly stoppered and allowed to cool in the antechamber of a dry box under vacuum before being transported into the dry box. HCF₂CF₂SO₃H (0.5 g) and 1-dodecene (30 ml) are added to the round bottomed flask in the dry box. The flask is then lowered into an oil bath and heated for 2 hours at 100° C. with stirring.

Upon completion of the reaction, the ionic liquid and acid form a separate phase at the bottom, with the product in the top phase. The product is colorless, i.e. water-white. GC analysis confirmed the conversion of the 1-dodecene to the equilibrium isomers, with less than 20% of the 1-dodecene remaining.

### Example 2 (Comparative Example)

### Isomerization of 1-dodecene in the Absence of an Ionic Liquid

Example 1 is repeated except that only catalyst is used. Ionic liquid is omitted. GC analysis shows that less than 5% of the 1-dodecene reacts. This is low conversion. Only one phase is observed after the reaction is ended. The color of the solution after the reaction is deep red. Depending on the intended use of the product, color formation is often not desirable.

1. A process for making internal olefins comprising:
(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid comprised of an ammonium cation and an anion having the Formula: 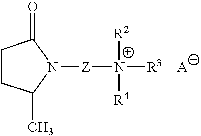
wherein:
(i) Z is —(CH₂)ₙ—, wherein n is an integer from 2 to 12;
(ii) R², R³ and R⁴ taken independently are H, —CH₃, —CH₂CH₃ or C₃ to C₆ straight-chain or branched monovalent alkyl; and
(iii) A⁻ is R⁵—SO₃⁻ or (R⁶—SO₂)₂N⁻; wherein R⁵ and R⁶ are independently selected from the group consisting of:
(a) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(b) —CH₃, —C₂H₅, or C₃ to C₂₅ straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(C) C₆ to C₂₅ unsubstituted aryl or unsubstituted heteroaryl having one to three heteroatoms independently selected from the group consisting of O, N and S; and
(d) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of:
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
thereby forming an isomer phase comprising at least one internal olefin and an ionic liquid phase that comprises the at least one acid catalyst; and
B) separating the isomer phase from the ionic liquid phase, thereby forming a separated ionic liquid phase.

2. The process of claim 1 wherein A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻.

3. The process of claim 1 wherein n is an integer from 2 to 6.

4. The process of claim 2 wherein n is an integer from 2 to 6.

5. The process of claim 1 wherein said at least one acid catalyst is selected from the group consisting of:
(i) bismuth triflate;
(ii) yttrium triflate;
(iii) ytterbium triflate;
(iv) neodymium triflate;
(v) lanthanum triflate;
(vi) scandium triflate;
(vii) zirconium triflate;
(viii) Formula (I); 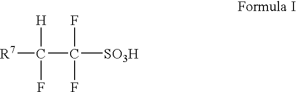
wherein:
R⁷ is selected from the group consisting of:
(1) halogen;
(2) —CH₃, —C₂H₅ or C₃ to C₁₅ straight-chain or branched alkane or alkene, optionally substituted with at least one member selected from the group consisting of Cl, Br, I, OH, NH₂ and SH;
(3) —OCH₃, —OC₂H₅ or C₃ to C₁₅ straight-chain or branched alkoxy, optionally substituted with at least one member selected from the group consisting of Cl, Br, I, OH, NH₂ and SH;
(4) C₁ to C₁₅ straight-chain or branched fluoroalkyl, optionally substituted with at least one member selected from the group consisting of Cl, Br, I, OH, NH₂ and SH;
(5) C₁ to C₁₅ straight-chain or branched fluoroalkoxy, optionally substituted with at least one member selected from the group consisting of Cl, Br, I, OH, NH₂ and SH;
(6) C₁ to C₁₅ straight-chain or branched perfluoroalkyl; and
(7) C₁ to C₁₅ straight-chain or branched perfluoroalkoxy;
(ix) Formula (II) 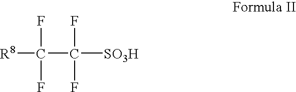
wherein:
R⁸ is selected from the group consisting of:
(1) —CH₃, —C₂H₅ or C₃ to C₁₅ straight-chain or branched alkoxy, optionally substituted with at least one member selected from the group consisting of Cl, Br, I, OH, NH₂ and SH;
(2) C₁ to C₁₅ straight-chain or branched fluoroalkoxy, optionally substituted with at least one member selected from the group consisting of Cl, Br, I, OH, NH₂ and SH; and
(3) C₁ to C₁₅ straight-chain or branched perfluoroalkoxy; and
(x) Formula (III); 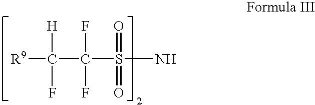
wherein:
R⁹ is selected from the group consisting of:
(1) halogen;
(2) —CH₃, —C₂H₅ or C₃ to C₁₅ straight-chain or branched alkane or alkene, optionally substituted with at least one member selected from the group consisting of Cl, Br, I, OH, NH₂ and SH;
(3) —OCH₃, —OC₂H₅ or C₃ to C₁₅ straight-chain or branched alkoxy, optionally substituted with at least one member selected from the group consisting of Cl, Br, I, OH, NH₂ and SH;
(4) C₁ to C₁₅ straight-chain or branched fluoroalkyl, optionally substituted with at least one member selected from the group consisting of Cl, Br, I, OH, NH₂ and SH;
(5) C₁ to C₁₅ straight-chain or branched fluoroalkoxy, optionally substituted with at least one member selected from the group consisting of Cl, Br, I, OH, NH₂ and SH;
(6) C₁ to C₁₅ straight-chain or branched perfluoroalkyl; and
(7) C₁ to C₁₅ straight-chain or branched perfluoroalkoxy.

6. The process of claim 5 wherein said at least one acid catalyst is selected from the group consisting of:
(i) 1,1,2,2-tetrafluoroethanesulfonic acid;
(ii) 1,1,2,3,3,3-hexafluoropropanesulfonic acid;
(iii) 2-chloro-1,1,2-trifluoroethanesulfonic acid;
(iv) 1,1,2-trifluoro-2-(perfluoroethoxy)ethanesulfonic acid;
(v) 1,1,2-trifluoro-2-(trifluoromethoxy)ethanesulfonic acid; and
(vi) 1,1,2-trifluoro-2-(perfluoropropoxy)ethanesulfonic acid

7. The process of claim 1 wherein the at least one acid catalyst is used at a concentration of from about 0.1% to about 20% by weight of the weight of the α-olefin(s) at the start of the reaction.

8. The process of claim 1 wherein the temperature is from about 50° C. to about 175° C.

9. The process of claim 1 wherein the reaction is carried out under an inert atmosphere at atmospheric pressure.

10. The process of claim 9 wherein the reaction is carried out under nitrogen, helium or argon.

11. The process of claim 1 wherein:
(i) n is an integer from 2 to 6;
(ii) A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻;
(iii) said at least one acid catalyst is selected from the group consisting of 1,1,2,2-tetrafluoroethanesulfonic acid and 1,1,2,3,3,3-hexafluoropropanesulfonic acid; and
(vii) the temperature is from about 50° C. to about 175° C.

12. The process of claim 1 wherein the separated ionic liquid phase is reused to form the reaction mixture.
